(19) United States
(12) Patent Application Publication
(10) Pub. No.: US 2025/0269162 A1
(43) Pub. Date: Aug. 28, 2025

(54) Core-Shell Microneedle Patches and Methods

(71) Applicant: Georgia Tech Research Corporation, Atlanta, GA (US)

(72) Inventors: Mark R. Prausnitz, Atlanta, GA (US); Wei Li, Atlanta, GA (US); Richard N. Terry, Atlanta, GA (US)

(21) Appl. No.: 18/859,731

(22) PCT Filed: May 17, 2023
(86) PCT No.: PCT/US2023/022560

§ 371 Date: Oct. 24, 2024

Related U.S. Application Data
(60) Provisional application No. 63/342,983, filed on May 17, 2022.

(51) Int. Cl. A61M 37/00

(52) U.S. Cl. CPC A61M 37/0015; A61M 2037/0023; A61M 2202/30; A61M 2209/088

(57) ABSTRACT

Microneedle patches that include a backing layer, and an array of microneedles extending from the backing layer. wherein the microneedles each include a core portion which includes a drug, and a shell portion and a cap portion, wherein the core portion is encapsulated within the shell portion and cap portion. The microneedles are configured to be inserted into mammalian tissue and then separate from the backing layer. The shell portion and/or the cap portion is biodegradable and delays release of the drug for an extended period.

### CROSS-REFERENCE TO RELATED APPLICATIONS

This application claims priority to U.S. Provisional Application No. 63/342,983, filed on May 17, 2022, which is incorporated herein by reference.

STATEMENT REGARDING FEDERALLY SPONSORED RESEARCH

This invention was made with government support under 7200AA20CA00016 awarded by the U.S. Agency for International Development. The government has certain rights in the invention.

### BACKGROUND

Conventional microneedle patches typically deliver a single bolus of a therapeutic or prophylactic agent into the skin upon penetration and dissolution of a water-soluble microneedle or a water-soluble microneedle coating. Such conventional microneedle-based delivery technologies therefore generally are unsuitable for long-term, sustained release of a drug.

While there are many conventional contraceptive methods, there remains a need for contraceptives that are safe, effective, and easy to access and self-administer. Non-hormonal contraception methods usually have high failure rates due to poor patient acceptance and compliance with correct use. Hormonal contraceptives, such as oral pills, transdermal patches, subcutaneous injections, implants, and intrauterine devices (IUDs), offer increased protection and improved patient compliance, but still have several shortcomings. For example, the efficacy of daily oral pill can be reduced because of improper use, but are easy to access and self-administer. Long-acting products, such as injections, implants, and IUDS, have improved efficacy but require expert administration and/or removal by healthcare providers, and therefore are harder to access. Currently, there are no long acting (e.g., offer protection for 6 months) contraceptives that are also safe, effective, and self-administered.

It therefore would be desirable to provide microneedles capable of long-term sustained drug release, particularly continuous, slow release or zero order release, for a variety of drugs, including but not limited to hormones, such as long-acting contraceptives.

It would also be desirable to provide new and improved methods for making microneedle arrays and patches comprising such arrays of microneedles capable of slow or sustained drug release.

### BRIEF SUMMARY

In one aspect, a microneedle patch is provided that includes: a backing layer; and an array of microneedles extending from the backing layer, the microneedles each comprising: a core portion which comprises a drug, and a shell portion and a cap portion, wherein the core portion is encapsulated within the shell portion and cap portion, wherein the microneedles are configured to be inserted into mammalian tissue, and separate from the backing layer, and wherein the shell portion and/or the cap portion is biodegradable and delays release of the drug for an extended period. The microneedles may be configured to release the drug via diffusion through the shell portion only, through the cap portion only, or through both the shell portion and the cap portion. In some embodiments, a microneedle patch is provided for administering a contraceptive agent or other drug, wherein the patch includes: a backing layer comprising an array of pedestals; and an array of microneedles extending from each of the pedestals, the microneedles each comprising: a core portion which comprises a biodegradable polymer in which a contraceptive agent or other drug is dissolved or dispersed, and a water insoluble shell portion and cap portion, wherein the core portion is enclosed within the shell portion and cap portion, wherein the microneedles are configured to be inserted into mammalian tissue, separate from the pedestals, and then release an effective amount of the contraceptive agent or other drug via diffusion through the shell portion only, through the cap portion only, or through both the shell portion and the cap portion, over an extended period of at least 30 days.

In another aspect, a method of making a microneedle patch is provided, wherein the method includes: (i) forming a plurality of microneedles in a mold, wherein each microneedle has a core portion comprising a drug, and a shell portion and a cap portion that fully surround the core portion; (ii) forming a backing layer connected to a base end of each of the plurality of microneedles; and then (iii) removing the backing layer and microneedles from the mold, thereby producing a microneedle patch. In a particular embodiment, the method includes: (i) casting a first composition in a mold having cavities defining a plurality of microneedles to form a shell portion in the mold; (ii) casting a second composition, which comprises a drug, into an interior space defined by the shell portion in the mold to form a core portion; (iii) casting a third composition onto the core portion in the mold to form a cap portion; and (iv) casting a fourth composition onto the cap portion in the mold to form a backing layer, thereby producing a microneedle patch comprising an array of microneedles extending from the backing layer, wherein each of the microneedles comprises the core portion, which comprises the drug, enclosed within the shell portion and the cap portion.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may or may not be present in various embodiments. Elements and/or components are not necessarily drawn to scale.

FIG. 1 is a perspective view of an array of core-shell microneedles, according to one or more embodiments of the present disclosure.

FIG. 2 is a schematic, cross-sectional, view of an array of core-shell microneedles, according to one or more embodiments of the present disclosure.

FIG. 3 depicts a process for manufacturing an array of core-shell microneedles, according to one or more embodiments of the present disclosure.

FIG. 4A depicts an array of core-shell microneedles prior to insertion into the skin, according to one or more embodiments of the present disclosure.

FIG. 4B depicts an array of core-shell microneedles after being inserted to the skin, prior to separating from the backing, according to one or more embodiments of the present disclosure.

FIG. 4C depicts an array of core-shell microneedles embedded within the skin after separating from the backing, according to one or more embodiments of the present disclosure.

FIG. 4D depicts drug diffusion from the core of the core-shell microneedles through the shell of the core-shell microneedles, according to one or more embodiments of the present disclosure.

FIG. 4E depicts drug diffusion from the core of the core-shell microneedles through the cap of the core-shell microneedles, according to one or more embodiments of the present disclosure.

FIG. 4F depicts drug diffusion from the core of the core-shell microneedles through the shell and the cap of the core-shell microneedles, according to one or more embodiments of the present disclosure.

FIG. 5A is a microphotograph showing a perspective view of an array of core-shell microneedles, according to one or more embodiments of the present disclosure.

FIG. 5B is a microphotograph showing a close-up of a core-shell microneedle, according to one or more embodiments of the present disclosure.

FIG. 5C is a microphotograph showing a close-up of a detached core-shell microneedle, with a magnified view of the cap, detached from the base, according to one or more embodiments of the present disclosure.

FIG. 6A is a graph comparing cumulative levonorgestrel (LGN) release, as a function of time, from a core-shell microneedle patch and a conventional microneedle patch, according to one embodiment of the present disclosure.

FIG. 6B depicts an exemplary core-shell microneedle, according to one or more embodiments of the present disclosure.

FIG. 6C depicts an exemplary conventional (core-only) microneedle, according to one or more embodiments of the present disclosure.

### DETAILED DESCRIPTION

Microneedles, microneedle arrays, and microneedle patches have been developed wherein the microneedles have a drug-containing core that, following in vivo insertion and separation from a microneedle patch, remains isolated (enclosed) within a shell and cap structure so that drug from the core of the separated microneedles is released into a patient's tissues slowly over an extended period. In particular, the shell (and optionally the cap) serve as a diffusion membrane that controls release of the drug. The shell and cap structure may be formed, for example, of one or more hydrophobic or water-insoluble polymers, which may be biodegradable.

The microneedles also are configured, e.g., have the geometry, composition, and mechanical properties, to be inserted into the skin or other biological tissues and then to separate from a backing layer of a microneedle patch as part of an administration method.

In some embodiments, the microneedles advantageously are able to control drug release for an extended period, which may be from 30 days to 1 year, e.g., at least 2 months, 3 months, 4 months, 5 months, or 6 months. Drug release may occur continuously over the extended period, or it may be delayed until the extended period has elapsed, which might be particularly useful as a vaccine booster.

In addition, methods have been developed for making arrays and patches of such microneedles. In a preferred embodiment, the methods include using a single mold to form all of the structural components of the microneedles and backing, which methods are simpler and most cost effective for large scale production than conventional methods for making microneedles having a drug core.

### Microneedle Patches

In some embodiments, the microneedle patch includes a backing layer; and an array of microneedles extending from the backing layer, wherein the microneedles have a core portion which comprises a drug, a shell portion, and a cap portion, wherein the core portion is enclosed (or encapsulated) within the shell portion and cap portion. The microneedles are configured to be inserted into mammalian tissue. In some preferred embodiments, the microneedles are configured to separate from the backing layer to remain embedded in the tissue. In preferred embodiments, the shell portion and/or the cap portion is biodegradable and delays release of the drug for an extended period.

In some preferred embodiments, the microneedles are configured to release the drug via diffusion through the shell portion only, the cap portion only, or through both the shell portion and the cap portion. Even if the shell or cap are biodegrading during the drug release process, the drug is considered to be released from the core by diffusion (i.e., through a biodegrading polymer shell and/or cap).

The shell portion and the cap portion may be formed of essentially any suitable biocompatible material that is substantially water insoluble so that they are able to isolate the core portion for an extended time in vivo, and preferably biodegrade while and/or after serving this function.

In some preferred embodiments, the shell and cap portions are formed of one or more biodegradable polymers. In some embodiments, the shell and cap portions may be formed of poly (L-lactide) (PLLA, also referred to as poly (L-lactic acid)) and/or polylactide (PLA, also referred to as poly (lactic acid) or poly (D,L-lactic acid)). Other suitable biodegradable polymers known in the art may also be used.

In some preferred embodiments, the backing layer includes one or more water-soluble materials, which facilitates separation of the microneedles from the backing layer following in vivo insertion of the microneedles and exposure of the backing layer to interstitial fluid or other aqueous fluids. In some embodiments, the backing layer includes polyvinyl pyrrolidone (PVP), polyvinyl alcohol (PVA), one or more sugars, such as sucrose, or a combination thereof. In some embodiments, the backing layer includes effervescent materials that will react and effervesce upon exposure of the backing layer to interstitial fluid or other aqueous fluids, which may accelerate separation of the microneedles from the backing layer. For example, the backing layer may include sodium bicarbonate, citric acid, or another an effervescent material, as described, for example, in U.S. Publication 2020/0238065 to Prausnitz et al.

In other embodiments, the interface of the backing layer and the cap portion includes a mechanical weakness so that the microneedles may “break apart” from the backing. For example, the mechanical weakness may be in the form of a cavity, or air bubble, that reduces the strength of the backing-cap interface. The microneedle patch may include a pedestal formed at the interface of the backing layer and each of the cap portions of the microneedles. The pedestal may be part of, e.g., integrally formed (e.g., molded) with, the backing layer.

The core portion may consist of one or more drugs, or, more often, includes one or more drugs dispersed in a suitable matrix material. The matrix material of the core may further control release of the drug, for example, serving as a rate limiting barrier to diffusion. In some embodiments, the matrix material of the core comprises a biodegradable polymer in which the drug is dissolved or dispersed. In some embodiments, the biodegradable polymer of the core includes poly (lactide-co-glycolide) (PLGA).

As noted above, the microneedles may be configured to separate from the backing layer. This separation may be triggered by a process that includes aqueous dissolution of a material forming at least part of the backing layer, as also described above. Alternatively or in addition, a mechanical shearing force, or an axial force, may be applied the backing layer following microneedle insertion, which causes a mechanical fracture between the microneedles and the backing layer, for example at an interface of the cap portion and the backing layer. In some embodiments, the microneedles are configured to separate within a period of 1 minute after the microneedles are inserted into the skin or other mammalian tissue. In some other embodiments, the microneedles are configured to separate within a period of between 1 minute and 10 minutes, such as between 2 minutes to 9 minutes, 3 minutes to 8 minutes, 4 minutes to 6 minutes, or preferably 5 minutes. In some further embodiments, the microneedles are configured to separate in less than 1 minute, such as 45 seconds, 30 seconds, 15 seconds 10 seconds, or preferably 5 seconds.

Based on the selected drug, the selected materials for the other structural components of the microneedle, and the thickness of the shell and cap portions, the release of the drug from the separated microneedle may be continuous over a period from 1 month to 1 year. As used herein. “continuous release” refers to any mechanism of drug release that occurs over the course of a predetermined period of time. For example, continuous release may be a constant release profile, a release profile where there is an initial burst of release, a period of inactivity, then constant release thereafter, or a variable release profile having short, periodic release bursts throughout the predetermined period. In some embodiments, the microneedles are configured to release the drug from the separated microneedle continuously over a period from 2 months to 6 months. In some embodiments, the drug comprises a steroid. In some embodiments, the drug comprises a hormone, such as levonorgestrel or another contraceptive hormone.

The drug may be one intended for systemic administration or effect, or it may be one targeting the tissues at the site of delivery. For example, the drug may be one effective to treat a dermatological condition, i.e., administering a drug into the skin in order to treat or prevent skin disorders, e.g., eczema, psoriasis, rosacea, acne, etc.

In some embodiments, the drug is a vaccine, such as used in the prophylaxis of infectious diseases such as influenza, measles, COVID, etc.

In some preferred embodiments, the shell portion and the cap portion are not formed of a composition containing a drug, such that the entire drug payload is located in the core portion at the time of manufacturing. In some cases, there may be a small amount of drug in the shell and cap even at the time of manufacturing but the goal (even after storage) in those cases is that the fraction of the drug in the shell and cap would less than 10%, less than 5%. or less than 2%, by weight. In some other embodiments, the shell portion and/or the cap portion also intentionally include a drug. That drug may be identical to or different from the drug in the core portion.

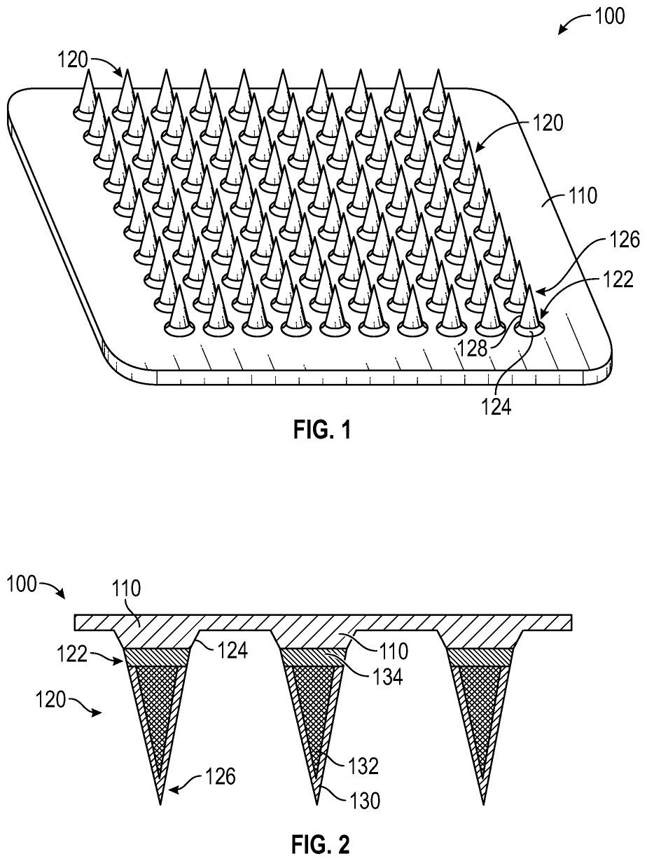

One example of a microneedle patch with an array of microneedles is depicted in FIG. 1. The microneedle patch 100 includes a backing layer 110 from which microneedles 120, in a 10×10 array, extend. The phrase “backing layer” and the terms “base substrate” or “substrate” are used interchangeably herein. Each microneedle 120 has a proximal end 122 attached to the backing layer 110 directly, or indirectly, via one or more proximal portions, or pedestals, 124, and a distal tip end 126 which is sharp and effective to penetrate biological tissue. The microneedle 120 has tapered sidewalls 128 between the proximal end 122 and distal end 126 of the microneedle 120. Other geometries are possible.

The microneedles of the microneedle patch are designed to insert into skin or another biological tissue and separate from the backing layer shortly after insertion. In some embodiments, the microneedles 120 and/or the backing layer 110, preferably the pedestal 124, include a feature, such as pre-fractured regions, porosity, bubble structures, effervescent material, and/or water-soluble material that facilitates separation of the microneedles 120) from the backing 110. As used herein with regard to the separation of the microneedles, the terms “facilitate”, “facilitating”, and the like refer to a feature that (i) reduces a minimum force (e.g., an axial or shearing force) necessary to achieve separation of the microneedles, (ii) reduces the amount of backing material that must dissolve in order to achieve separation of the microneedles (for example, a bubble structure may result in thinner walls in a microneedle), (iii) increases the rate of dissolution of the backing layer to which the microneedles are attached, or (iv) a combination thereof.

Upon separation, the microneedles may be embedded in a biological tissue, such as a patient's skin. A microneedle is “embedded” in a biological tissue when all or a portion of the microneedle's structure is below the surface of the biological tissue. In some embodiments, all of the embedded microneedle's structure is below the tissue surface.

In some embodiments, the backing layer 110, or more preferably the pedestal 124, includes an effervescent material to facilitate separation of the microneedles from the backing. As used herein, the phrase “effervescent material” refers to a material or combination of materials that generate a gas upon contacting an aqueous liquid. When a microneedle patch includes an effervescent material, the effervescent material may react when contacted with an aqueous liquid, such as a biological fluid (e.g., an interstitial fluid) on, in, or under a biological tissue, thereby generating a gas to expedite dissolution of the backing. In some preferred embodiments, the backing layer 110 includes polyvinyl pyrrolidone (PVP) with ethanol, sodium bicarbonate, and citric acid in order to generate effervescence that facilitates separation of the microneedles from the backing.

In some embodiments, the backing layer 110 includes a water-soluble material effective to facilitate separation of the microneedles from the backing. As used herein, the phrase “water-soluble material”, “hydrophilic material”, or the like refers to a material or combination of materials that dissolves, or substantially dissolves, upon contacting an aqueous liquid. When the microneedle patch includes a water-soluble material, the water-soluble material may make contact with an aqueous liquid, such as a biological fluid (e.g., an interstitial fluid) on, in, or under a biological tissue, thereby partially or fully dissolving at least a portion of the backing. In some preferred embodiments, the backing layer 110 includes PVA and sucrose.

In some other embodiments, the interface of the backing layer and the cap portion includes a mechanical weakness so that the microneedles may “break apart” from the backing. For example, the mechanical weakness may be in the form of a cavity, or air bubble, that reduces the strength of the backing-cap interface.

As shown in FIG. 2, microneedle patch 110 includes microneedles 120, each of which includes a shell portion 130, a core portion 132, and a cap portion 134 adjacent to the backing layer 110. The shell portion 130 may surround the core portion 132 on most sides, and the cap portion 134 covers the core 132 where the shell 130 does not, such that the shell 130 and cap 134 together surround the core region 132. In some preferred embodiments, the core 132 may be in the shape of a cone, such that the shell 130 is in the shape of a hollow cone and the cap 134 is in the shape of a truncated cone. While it is preferred that the shell 130 and the cap 134 entirely surround the core 132, it would be understood that the shell 130 and/or cap 134 may have gaps, cracks, or other imperfections such that the shell 130 and cap 134 do not fully encase the core 132.

In some embodiments, the length of the microneedles may be between about 50 μm and 2000 μm, from about 100 μm to about 2000 μm, from about 100 μm to about 1500 μm, from about 200 μm to about 1000 μm, or ideally, between about 500 μm and 1000 μm. In some embodiments, the array of microneedles includes from 10 to 1000 microneedles, from 10 to 500 microneedles, from 10 to 250 microneedles, from 50 to 250 microneedles, or 100 microneedles (e.g., a 10-by-10 array of microneedles).

In some embodiments, the length of the pedestal 124 may be between about 50 μm and 1000 μm, from about 100 μm to about 750 μm, from about 100 μm to about 500 μm. from about 200 μm to about 400 μm, or from about 500 μm and 1000 μm.

In some embodiments, the shell 130 and cap 134 include a biocompatible polymer having an expected degradation time of longer than six months. In some embodiments, the biocompatible polymer is hydrophobic and/or non-water soluble. For example, the biocompatible polymer may be polylactide (PLA), poly-1-lactic acid (PLLA), poly (lactide-co-glycolide) (PLGA), and other biodegradable polymers and copolymers. As used herein, the term “non-water soluble” refers to materials that do not substantially dissolve or lose structural integrity upon contact with an aqueous liquid, unless they subsequently biodegrade to yield water soluble degradation products. In some preferred embodiments, the shell 130 and the cap 134 include different biocompatible polymers. For example, the shell 130 includes PLLA and the cap 134 includes PLA.

In some embodiments, the core portion 132 includes a drug and a biodegradable polymer having an expected degradation time of less than six months. In some embodiments, the biocompatible polymer is a hydrophobic polymer, such as poly (lactide-co-glycolide) (PLGA), polylactide (PLA), poly-1-lactic acid (PLLA), and other biodegradable polymers and copolymers.

A wide range of drugs may be formulated for delivery to biological tissues using the core-shell microneedle patches disclosed herein. As used herein, the term “drug” refers to a prophylactic, therapeutic, or diagnostic agent useful in medical applications, as well as agents used cosmetic, cosmeceutical, tattoo, and other non-medical applications. It may be any suitable active pharmaceutical ingredient or allergen. In some embodiments, the drug may be selected from small molecules and larger biotechnology produced or purified molecules (e.g., peptides, proteins, DNA, RNA, aptamers). In some preferred embodiments, the drug is a hormone or a steroid. The hormone may include a contraceptive hormone such as levonorgestrel, etonogestrel, nestorone and other progestins, as well as estrogen, estradiol and other estrogens. In some embodiments, the drug has a dermatological indication. In some embodiments, the drug comprises a vaccine. Examples of vaccines include vaccines for infectious diseases, therapeutic vaccines for cancer, neurological disorders, allergies, and smoking cessation or other addictions.

In some embodiments, the microneedles within a given array of microneedles all contain the same drug. In some other embodiments, the microneedles within a given array of microneedles may contain different drugs. For example, the drugs may be different in each microneedle, in different rows of microneedles, or in sections/regions of the microneedle array. For example, the drugs may be different in the core, shell and/or cap of a microneedle.

In some preferred embodiments, the core portion 132 is designed to continuously release the drug from the microneedles via diffusion through the shell portion 130 and/or cap portion 134 over an extended period of time at a controlled rate. In some embodiments, the extended period of time is one month, 2 months, 3 months, 4 months, 5 months, 6 months, one year, or greater. In some embodiments, the release rate is substantially zero-order, which is achieved by diffusion across a rate-controlling membrane that is provided by the shell portion and/or the cap portion of the microneedles. In some other embodiments, the release rate is substantially first-order. In further embodiments, the release is pulsatile over the duration of the release period (e.g., a single bolus is administered daily over the course of the extended treatment period).

The microneedle patch may further include other structural elements (not shown in FIG. 1) that enhance storage and usability of the microneedles. For example, the patch may include a housing and/or other layers, such as a handle layer, on the side of the backing layer opposing the microneedles. Examples of such other additional structural components are described in U.S. Pat. No. 10,265,511 to McAllister et al., which is incorporated herein by reference.

### Methods of Making Microneedle Arrays, Microneedle Patches

The microneedles described herein may be made by any suitable process. However, in some preferred embodiments, arrays of the microneedles are made using a molding process, which advantageously is highly scalable. In a particularly preferred embodiment, the fabrication process includes a series of solution casting steps carried out within the same mold, which lends itself to low-cost mass production. This contrasts with efforts by others to make core-shell microneedles, in which the shell, the core, the cap, and the patch backing layers are each individually fabricated using separate molds and processes, and then assembled into a complete microneedle patch using a process requiring many more steps.

The filling and molding steps described herein may be referred to as “casting”. In some embodiments, the casting methods, molds, and other equipment may be adapted from those known in the art, such as described in U.S. Pat. No. 10,828,478 to McAllister et al., which is incorporated herein by reference. The methods for making the microneedles preferably are performed under a minimum ISO 7 (class 10,000) process or an ISO 5 (class 100) process).

In a particular embodiment, a method of making a microneedle patch includes: (i) forming a plurality of microneedles in a mold, wherein each microneedle has a core portion comprising a drug, and a shell portion and a cap portion that surround the core portion; (ii) forming a backing layer connected to a base end of each of the plurality of microneedles; and then (iii) removing the backing layer and microneedles from the mold, thereby producing the microneedle patch. The method may further include forming an array of pedestals for each of the microneedles. For example, the mold may include a funnel shape portion near the opening such that the pedestals (i.e., the funnel shape portions) are formed simultaneously with formation of the backing layer. In this way, the backing layer comprises an array of pedestals, each being connected to a base end of one of the plurality of microneedles.

In a particular embodiment, a method for making the microneedles includes: (i) casting a first composition in a mold having cavities that define a plurality of microneedles to form a shell portion in the mold; (ii) casting a second composition, which comprises a drug, into an interior space defined by the shell portion in the mold to form a core portion; (iii) casting a third composition onto the core portion in the mold to form a cap portion; and (iv) casting a fourth composition onto the cap portion in the mold to form a backing layer, thereby producing a microneedle patch comprising an array of microneedles extending from the backing layer, wherein each of the microneedles comprises the core portion, which comprises the drug, enclosed within the shell portion and the cap portion. In some preferred embodiments, the first, second, third, and fourth compositions each includes a fluid comprising a polymeric structural material dissolved in a solvent, wherein the solvent of each of the second, third, and fourth compositions is substantially a non-solvent for the polymeric structural material of the first, second, and third compositions, respectively. For example, the polymeric structural material of the first, second, and third compositions may each be a different biodegradable polymer, and the polymeric structural material of the fourth composition may be a water-soluble polymer, such that a different solvent may be selected for each polymer that does not dissolve or degrade the polymeric structural portion of the microneedle formed in a preceding step. The solvents in the composition may be substantially non-solvents for the drug present in the second composition.

Centrifugation, drying, and/or vacuum may be used to aid in the steps that form each of the portions of the microneedle patch. For example, centrifugation and/or vacuum may help evenly disperse the first composition within the mold to form a shell and/or remove any excess of the third composition so that the microneedles are uniformly capped.

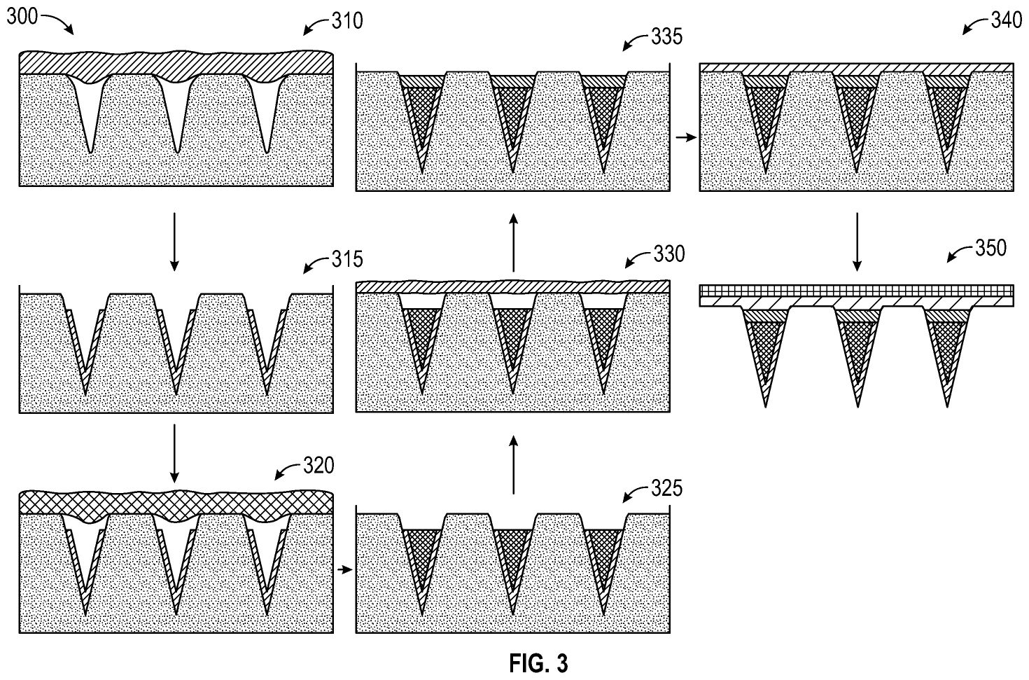

An example of a manufacturing process 300 for making the core-shell microneedles described herein is shown in FIG. 3. It includes at least five steps: (1) the first cast filling (310) to form the shell portion, (2) the second cast filling (320) to form the core portion, (3) the third cast filling (330) to form the cap portion, (4) the fourth cast filling (340) to form the backing layer, and (5) demolding the produced microneedle patch (350). The mold is a negative of the microneedles, and at least part of the backing layer. Suitable microneedle molds typically include an array of between 10 and 1000 microneedles and may be made of silicone or other elastomeric materials, as known in the art.

To form a shell portion of the microneedles, a first cast composition is prepared and then transferred onto the mold (310). This may be referred to as the first cast. In some embodiments, the first cast is formed by dissolving or dispersing a suitable structural material for the shell in a suitable solvent to form a castable fluid. For example, the structural material preferably is a biodegradable polymer and the solvent may be an organic solvent. In one embodiment, the biodegradable polymer is PLLA (L-lactide, ester end-capped) and the solvent is dioxane, or another suitable solvent capable of swelling into the mold to form a “skin” of the first cast on the mold cavity walls. The mold with the first cast then is centrifuged to evenly distribute and coat the mold with the first cast to form a film which is the uniform shell portion (315). The mold and film typically undergo drying (e.g., with heat and vacuum) to remove the solvent and convert the film to a solid shell.

Next, to form a core portion of the microneedles, a second cast composition is prepared and then transferred onto the mold (320). This may be referred to as the second cast. In some embodiments, the second cast is formed by dissolving or dispersing a drug and a suitable structural material for the core in a suitable solvent to form a castable fluid. For example, the structural material may be a biodegradable polymer and the solvent may be an organic solvent, or an aqueous organic solvent mixture. In one embodiment, the biodegradable polymer is 85/15 lactide/glycolide molar ratio, ester end-capped) and the solvent is diglyme/water (95%/5%, v/v). The mold with the second cast then is centrifuged to pull the cast material into the mold and the interior of the shell (325). The mold and film typically undergo drying (e.g., with heat and vacuum) to remove the solvent and solidify the core portion. In this case, the second-cast solvent (diglyme/water) is substantially a nonsolvent for the PLLA shell created by the first cast.

Next, to form a cap portion of the microneedles, a third cast composition is prepared and then transferred onto the mold (330). This may be referred to as the third cast. In some embodiments, the third cast is formed by dissolving or dispersing a suitable structural material for the cap in a suitable solvent to form a castable fluid. For example, the structural material may be a biodegradable polymer and the solvent may be an organic solvent. In one embodiment, the biodegradable polymer is PLA (DL-lactide, ester end-capped) and the solvent is dimethylacetamide. The mold with the third cast then is centrifuged to pull the cast material into the mold and remove any excess of the third cast so that the microneedles are uniformly capped (335). The mold and film typically under drying (e.g., with heat and vacuum) to remove the solvent and solidify the cap portion. In this case, the third-cast solvent (dimethy lacetamide) is substantially a nonsolvent for the PLLA shell formed by the first cast.

Next, to form a backing layer for the microneedle patch, a fourth cast composition is prepared and then transferred onto the mold (340). This may be referred to as the fourth cast. In some embodiments, the fourth cast is formed by dissolving or dispersing a suitable structural material for the backing layer in a suitable solvent to form a castable fluid. For example, the structural material may comprise one or more water-soluble materials and the solvent may be an aqueous solvent or solvent mixture. In one embodiment, structural material is a combination of PVA and sucrose to yield a desired combination of mechanical strength and water solubility. In another embodiment, polyvinyl pyrrolidone (PVP) can be formulated in ethanol with sodium bicarbonate and citric acid that generates effervescence that expedites microneedle separation from the backing layer upon contacting aqueous interstitial fluid in the skin. Anhydrous ethanol is a suitable solvent because it dissolves citric acid and supports a suspension of sodium bicarbonate, but does not allow their water-based effervescence reaction to occur during fabrication. PVP may serve as a suitable matrix material for the backing layer because it is soluble in ethanol during manufacturing and soluble in water during application of the microneedle patch to skin.

The mold with the fourth cast then is dried (e.g., with heat and vacuum) to remove the solvent and solidify the backing layer. In this case, the fourth-cast solvent (water or ethanol) is substantially a nonsolvent for all three polymers of the microneedle, i.e., the PLA found in the third cast, the PLGA in the second cast, and the PLLA found in the first cast.

After the final cast is substantially dried, the core-shell microneedle patch is removed from the mold, i.e., demolded (350).

As noted above, each of the casts may include solvents with different solubility properties in order to prevent dissolution of the previously cast composition during fabrication of the microneedle patches. In some embodiments, the solvents of the second, third, and fourth casts must not dissolve the polymer of the first cast. In some embodiments, the first cast solvent must differ from at least the second cast solvent, the second cast solvent must differ from at least the first and the third cast solvents, the third cast solvent must differ from at least the second and the fourth cast solvent, and the fourth cast solvent must differ from at least the third cast solvent. In some other embodiments, each cast includes a different solvent. Generally, it is immaterial whether or not the fourth cast dissolves the second cast, because they should not contact each other. However, in a preferred embodiment, the fourth cast does not dissolve the second cast, but in principle it could, as long as it does not dissolve the first or third casts.

In some other embodiments, one or more of the casts are formed without the use of a solvent, by using melted compositions, e.g., wherein the structural polymeric material of the shell, core, cap, or backing layer portions is cast at a temperature above its melting point and then cooled/solidified to form that portion of the microneedle patch. However, the temperature of the casts should not be high enough to cause degradation of or damage to the drug comprised in the second cast. For example, a biocompatible polymer with a lower melting point, such as polycaprolactone (PCL) or copolymers thereof, may be preferred for use in the second cast, because PCL advantageously compounds with drugs at a lower temperature.

In some such cases, each cast may have different melting points. For example, the first cast may have the highest relative melting point, the second cast must have a melting point lower than that of the first cast but higher than the third cast, the third cast must have a melting point lower than that of the second cast but higher than the fourth cast, and the fourth cast must have the lowest relative melting point. In these embodiments, the melting points of at least the first cast and the third cast must be high enough to prevent melting upon insertion, i.e., the melting points of the first and third casts must be above body temperature, in order to maintain the solid structure of the shell and cap during the extended period of in vivo drug release by diffusion of drug through the shell (and cap).

In some other embodiments, a combination of melting cast solutions and solvent cast solutions may be used. For example, one cast may be formed of a melting composition and the remaining three casts may be formed with a solvent, two casts may be formed of a melting composition and the remaining two casts may be formed with a solvent, or three casts may be formed with a melting solution and the remaining cast may be formed with a solvent.

### Methods of Use

The microneedle arrays described herein may be used to administer a variety of substances into a biological tissue site in a human or other mammal. As used herein, the phrase “biological tissue” generally includes any human or mammalian tissue. The biological tissue may be the skin or a mucosal tissue of a human or other mammal in need of treatment, or prophylaxis or cosmetic enhancement. In a preferred embodiment, the method includes applying a microneedle patch that includes an array of the core-shell microneedles to a skin surface in a manner to cause the microneedles to penetrate the stratum corneum and enter the viable epidermis and, possibly, the dermis. It is envisioned, however, that the present devices and methods may also be adapted to other biological tissues and other animals.

As used herein, the phrase “penetrate a tissue surface” includes penetrating a biological tissue surface with at least the distal tip ends of the microneedles. In some embodiments, upon separation of a microneedle from the backing layer, a proximal end of the microneedle may be above a tissue surface, substantially level with a tissue surface, or preferably below a tissue surface.

The microneedle patches may be self-administered or administered by another individual (e.g., a parent, guardian, or healthcare worker).

The methods described herein further include a simple and effective method of administering a drug to a patient with a microneedle patch. The method may include identifying an application site and, preferably, sanitizing the area prior to application of the microneedle patch (e.g., using an alcohol wipe). The microneedle patch is then applied to the patient's skin/tissue and manually pressed into the patient's skin/tissue (e.g., using the thumb or finger) or using a device to facilitate patch application so that the microneedles penetrate the tissue surface.

After administration, the backing layer (and any remaining microneedle patch structure) may be removed from the patient's skin or other tissue surface.

In some preferred embodiments, the microneedle patches described herein are used to administer a drug into the skin of patient over an extended period. In some embodiments, the method of administration includes inserting the microneedles of the patch across the stratum corneum (outer 10 to 20 microns of skin that is the barrier to transdermal transport) and into the viable epidermis and possibly the dermis. The small size of the microneedles enables them to cause little to no pain and target the intracutaneous space. The intracutaneous space is highly vascularized in the dermis and rich in immune cells in the dermis and epidermis. and provides an attractive path to administer vaccines and other prophylactic, therapeutic and cosmetic agents. In some preferred embodiments, the drug diffuses from the core portion of the inserted microneedle through the shell and/or cap portion(s) into the intracutaneous space to release the drug into interstitial fluid within the skin.

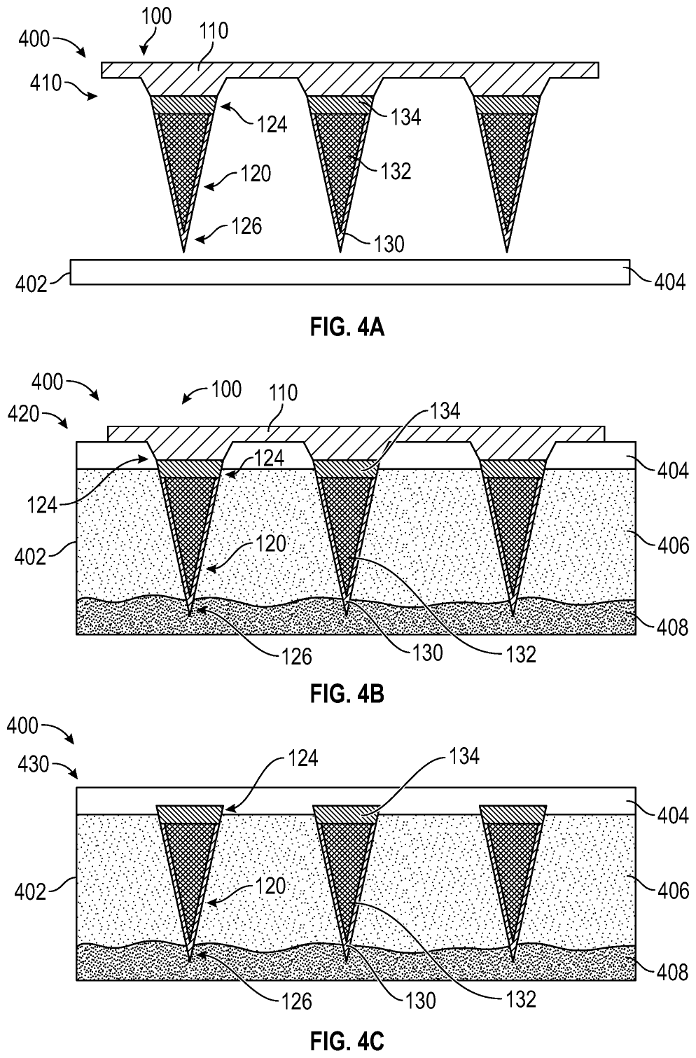

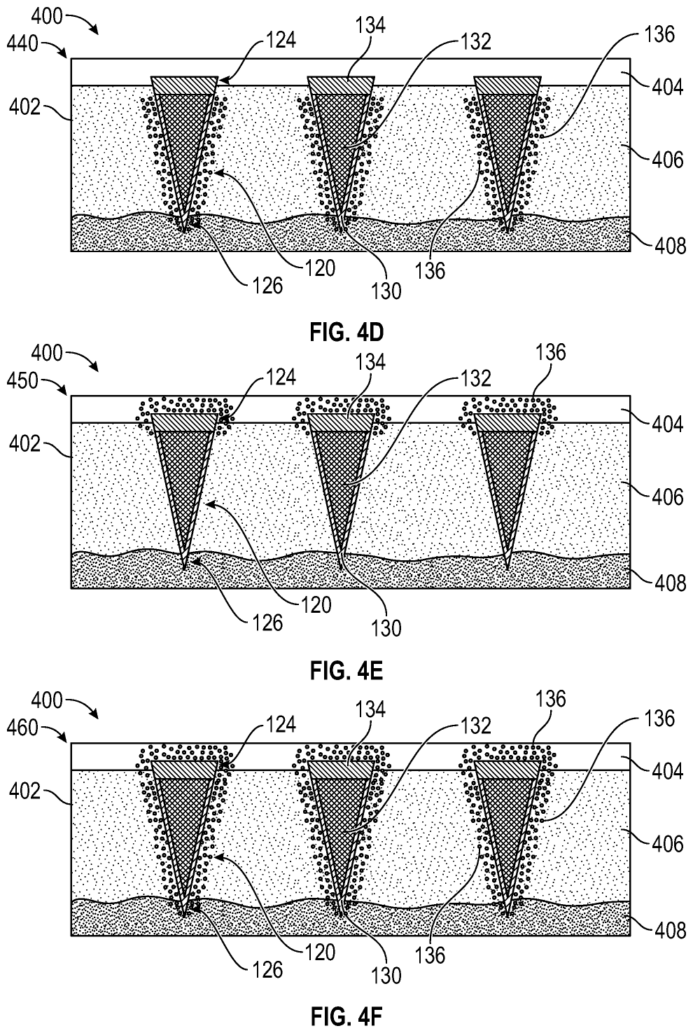

FIGS. 4A-4F illustrate an example of a method (400) for administering a drug into a patient's skin using the microneedles described herein. The method includes providing a microneedle patch 100 as described herein, aligning the microneedle patch 100 with a target site at the skin 402 surface (410), and inserting the microneedle patch into the skin 402 (420) to force the microneedles 120 through at least the stratum corneum 404 and into the viable epidermis 406 and possibly the dermis 408 layers of the skin. Subsequently, the microneedles 120 are separated from the backing layer 110 (430) such that the microneedles remain embedded within the skin 402. Separation may occur, for example, quickly after insertion when the backing layer 110 and/or proximal ends 122 of the microneedles are formed of fracturable, water-soluble and/or effervescent materials, enabling removal of the backing 110 within a few seconds or minutes after microneedle insertion. While the microneedles 120 remain within the skin 402, the drug 136 diffuses from the core portion 132 through only the shell portion 130 of the microneedles (440) as in FIG. 4D, only the cap portion 134 of the microneedles 120 (450) as shown in FIG. 4E, or through both the shell 130 and cap 134 portions (460) of the microneedles as shown in FIG. 4F.

The shell, cap, and other non-drug materials (e.g., matrix materials) of the core portions may biodegrade at rates that do not negatively impact the controlled rate of diffusion of drug therethrough. In this way, once the drug from the microneedles has been released. the remaining shell, cap, and matrix materials of the core portions will finish biodegrading.

Although the microneedles are shown, and often preferred, to separate from the backing before the drug is released from the core, this is not necessary. For example, the drug may begin to diffuse from the core through at least the shell and/or cap before and/or during their separation from the backing, or the microneedles may not separate from the backing at all.

This invention can be further understood with reference to the following non-limiting examples.

### Example 1. Design and Fabrication of Core-Shell Microneedle Patches for LGN Release

To make core-shell microneedle patches for months-long release of the contraceptive hormone levonorgestrel (LNG), high molecular weight PLLA (L-lactide, ester end-capped; inherent viscosity, 0.9 dL/g) was selected as the rate-controlling shell material because of its known crystallinity and very slow biodegradation rate, which should enable it to provide a stable diffusional barrier for controlled release of LNG from the core. PLGA (85/15) was selected as the core polymer, because this PLGA is biodegradable on a time-scale consistent with months-long release of encapsulated LNG, has sufficient mechanical strength for microneedles, and can be formulated using solvents that would not dissolve the PLLA shell during fabrication. LNG was selected as the contraceptive hormone in the microneedle core because of potency as a contraceptive hormone, its low solubility in water, and because it has been widely used in marketed long-acting contraceptive products with good safety and efficacy profiles. PLA (poly (D,L-lactide, ester end-capped; inherent viscosity, 0.5 dL/g) was selected as the polymer for the microneedle cap because it has a slow degradation rate, has sufficient mechanical strength for microneedles, and could be formulated using solvents that would not dissolve the PLLA shell during fabrication.

Polydimethylsiloxane (PDMS) (Dow Corning, Midland, MI) molds were used to fabricate the microneedle patches. In each PDMS mold, 112 microneedle cavities were arranged in a round array with a diameter of 1.1 cm. The center-to-center interval between the microneedles was 900 μm, and each microneedle cavity was conical with a base radius of 150 μm, a height of 600 μm, and a tip radius of approximately 10 μm. Above each microneedle cavity was an area for the patch backing, which contained an array of pedestals, each having a base diameter of 600 μm, a top diameter of 150 μm, and a height of 350 μm, that were positioned at the base of each microneedle to elevate the microneedles above the base of the backing, i.e., the base structure of the microneedle patch.

The fabrication process was designed as a series of solution casting steps onto a single silicone (PDMS) mold, as described with respect to FIG. 3. To fabricate core-shell microneedle patches, three solutions were cast sequentially to make the shell, core, and cap structures of the microneedles.

The first solution contained 1% (w/v) PLLA in dioxane. To prepare this solution, 0.1 g of PLLA (L-lactide, ester end-capped; inherent viscosity, 0.9 dL/g; Durect, Birmingham, AL) was dissolved in 10 mL dioxane (Sigma-Aldrich, St. Louis, MO). Dioxane was selected as the primary solvent because it is a good solvent for PLLA and it is able to swell into the PDMS mold, which helps to form a “skin” of PLLA on the mold cavity walls. In this way, shell structures may be formed on the inner surface of the mold cavities without allowing the PLLA to accumulate substantially in the tips of the needle cavities. Twenty microliters of this first casting solution were applied to the surface of the microneedle mold and then centrifuged at 3200 g for 20 minutes to form a film of PLLA in the mold that would become the outer shell of the microneedles.

Next, a second casting solution was prepared for the core consisting of 8% (w/v) solids, PLGA/LNG (60%/40%, w/w) in diglyme/water (95%/5%, v/v). To make this solution, 0.096 g of PLGA (D,L-lactide, ester end-capped; 85/15 lactide/glycolide molar ratio; inherent viscosity, 0.5 dL/g; Durect) and 0.064 g of LNG (Chemo Industriale Chimica S.R.L., Saronno, Italy) were dissolved in a mixture of 1.5 mL of diglyme (Sigma-Aldrich) and 0.5 mL of tetrahydrofuran (THF. Thermo Fisher Scientific, Waltham. MA), and then LNG was precipitated by slow evaporation of the THF to produce a colloidal suspension of LNG particles within the PLGA solution. For example, as described in U.S. Publication 2022/0401715, precipitation of LNG in the second casting solution may help prevent migration of soluble LNG into the shell and/or cap, where it could cause a more rapid release of LNG after insertion of the microneedle patch into the tissue. After evaporation of essentially all of the THF, additional diglyme and deionized (DI) water were added to obtain the final casting solution, which comprised LNG, PLGA, diglyme and water.

Fifteen microliters of the second cast solution were applied to the top of the PDMS mold within the shells and then centrifuged at 3200 g for 20 minutes to form the core part of the microneedles in the mold. 20 L of diglyme/water (95%/5%, w/w) was then pipetted on top of the mold, and after waiting 5 minutes, the mold was centrifuged at 3200 g for 20 minutes to wash residual casting solution on top of the mold into the mold cavities. After that, the mold was dried in a 60°° C., oven with vacuum for 12 hours.

A third casting solution for the cap contained 3.5% (w/v) PLA (D,L-lactide, ester end-capped; inherent viscosity, 0.5 dL/g; Durect) in dimethylactamide. Dimethylacetamide was used as the solvent because it is a good solvent for the medium-viscosity D,L-PLA used in the cap, but not for the high-viscosity PLLA used in the shell. Furthermore, its solubility properties, low volatility, and low mold-swelling tendency (i.e., unlike dioxane) minimized the formation of a PLA film on the PDMS mold surface above the microneedle cap during the casting process, which is important for allowing detachment of the microneedle after insertion into the skin.

This solution was applied on top of the dried mold, which was then centrifuged at 3200 g for 20 minutes to form the cap and seal the core within the cap and shell. Sealing the core-shell structure is effective to eliminate any holes or gaps that would enable leakage of LNG from the core during the slow release process. After that, the mold was put in a 60° C. oven with vacuum for 12 hours for another drying cycle.

As a final fabrication step, 80 μL of backing solution consisting of 18% (w/v) PVA (molecular weight: 6 kDa; Sigma-Aldrich) and 18% (w/v) sucrose (Sigma-Aldrich) dissolved in DI water, was applied to the dried PDMS mold surface to form the patch backing.

After air-drying for 3 hours, the mold was transferred to a desiccator for 2 days, followed by demolding with adhesive tape.

### Example 2. Characterization of Core-Shell Microneedle Patches

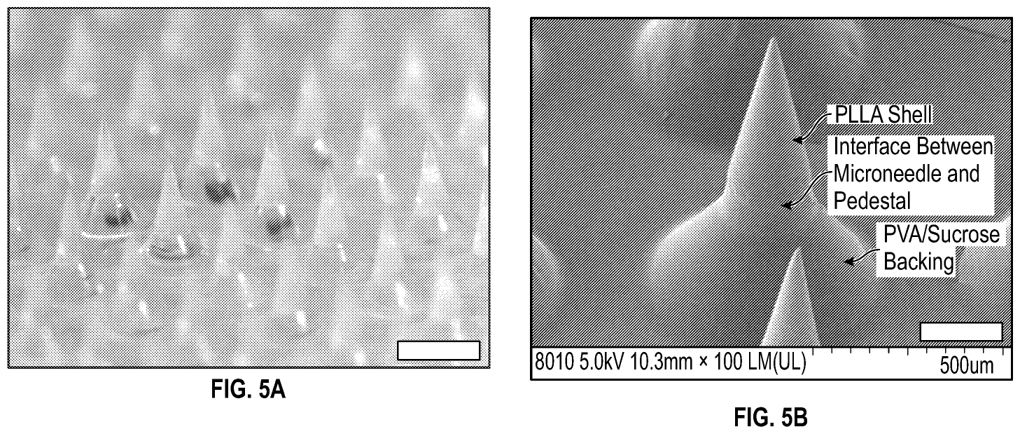

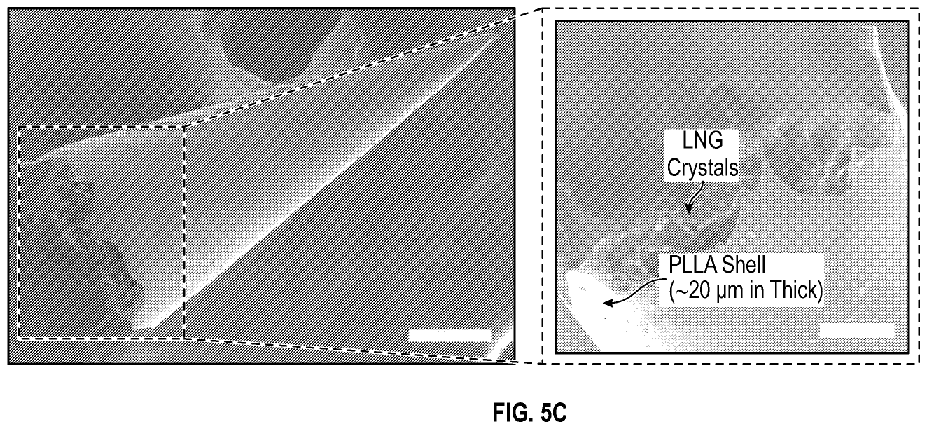

Core-shell microneedles have a conical shape connected to a tapered pedestal at the base, which is designed to facilitate deep microneedle insertion into the skin, as shown in FIGS. 5A-5C. Closer examination by scanning electron microscopy (SEM) showed an interfacial layer between the conical microneedle and the pedestal, which is the PLA cap, as well as a thin layer covering the outer microneedle surface, which was the PLLA shell. Inside the microneedles, there was a mixture of LNG crystals and PLGA polymer that formed the core part of microneedles acting as the drug reservoir. In this way, the microneedle core comprising LNG and PLGA polymer was fully encased by the outside shell-cap polymer pocket made of PLLA/PLA. A LNG loading of 0.28±0.01 mg per patch was determined by ultra-performance liquid chromatography (UPLC) of microneedles after dissolution in acetonitrile.

To further examine the core-shell microneedle structure, confocal microscopy revealed a core labeled with red fluorescence using Nile red dye surrounded by a shell and cap labeled with green fluorescence using fluorescein isothiocyanate (FITC). This imaging further validated the core-shell microneedle structure shown by SEM and designed by the fabrication procedure.

As an additional test to investigate whether LNG was localized in the microneedle core, confocal Raman microscopy was used to compare the compositional distribution in core-shell microneedles to microneedle cores fabricated without a shell.

A microneedle patch was cut in half with a razor blade and incubated in 50 mL water for 15 minutes to dissolve the water-soluble backing. Microneedle tips were collected and placed on a glass slide covered with aluminum foil, and dried in a desiccator for at least 4 days, followed by cutting microneedles perpendicularly to their central axis with a razor blade to obtain cross sections. The cross sections were then mapped with confocal Raman microscopy (inVia Qontor, Renishaw, Wotton-under-Edge, UK) at an excitation wavelength of 785 nm operated at 300 mW with a 1 second exposure time. Raman spectra from 1050.08 cm⁻¹ to 2083.76 cm⁻¹ were collected every 2 μm along the x- and y-axis using a 50× long working distance objective. Characteristic LNG peaks were observed between 1600 cm⁻¹ and 1700 cm⁻¹, and characteristic PLA and PLGA peaks were observed at 1700 cm⁻¹. The spectra were processed and peak sizes were quantified by signal-to-baseline ratio and converted to false color images with WiRE 5 software (Renishaw, New Mills, UK). At least 6 microneedle tips were examined in each experimental group.

In the core-only microneedles, LNG and PLGA were distributed throughout the microneedles. In contrast, LNG was encapsulated in the core section of the core-shell microneedles, and was surrounded by an outer layer of PLLA/PLA that formed the shell and cap, and also included LNG. Raman microscopy images also confirmed a shell thickness of approximately 20 μm.

It was difficult to distinguish the Raman spectra signals between PLLA, PLA, and PLGA because these polymers exhibited similar signal strength at the 1770 cm⁻¹ peak, and they are therefore reported as a combined PLGA/PLA signal. The PLGA Raman signal in the core of the core-shell microneedles appeared weaker than the PLGA signal in the center of the core-only samples. However, quantitative analysis of the PLGA signal values in the cores were similar in the core-shell and core-only samples. The apparently weaker PLGA signal in the core of core-shall samples can be explained by the strong PLLA/PLA signal generated by the dense shell, which made the PLGA core appear weaker due to autocalibration of signal intensity to avoid saturation of the PLLA/PLA signal of the shell.

### Example 3. Sealing of the Core in Core-Shell Microneedle Patches

Slow release from core-shell microneedles is a result of efficient encapsulation of drug inside the shell-cap pocket. To evaluate the sealing efficacy of core-shell microneedles in vitro, the second cast solution used to make the core part of the microneedles was made using a solution of 18% (w/v) PVA and 1% (w/v) fluorescein isothiocy anate-labeled bovine serum albumin (FITC-BSA) dissolved in phosphate-buffered saline (PBS), while keeping the shell and cap formulations the same. Each microneedle patch was put into 20 mL PBS, and the morphology and fluorescence of the microneedles was imaged using bright-field and fluorescence optics by fluorescence microscopy (SZX16, Olympus, Tokyo, Japan) at 0 hours, 0.5 hours, 1 hour, 3 hours, 6 hours, 12 hours, 24 hours, 48 hours, 72 hours, and 96 hours.

The fluorescence intensity of the FITC-BSA in the microneedle cores was strongly retained in PBS for 1 hour, after which it gradually dimmed for 3-4 days. In contrast, microneedles made of the same core material either with a shell but no cap or with neither a cap nor a shell dissolved in 25 minutes or 2 minutes, respectively. This demonstrates the significant diffusional barrier created by the shell and cap around the core.

### Example 4. Insertion of Core-Shell Microneedle Patches into Skin Ex Vivo

To evaluate the penetration, detachment, and delivery efficiency of the microneedle patches, the patch backing was changed to an effervescent backing made of 13% (w/v) PVP with two molecular weights (360/55 kDa, 50/50%, w/v; Sigma-Aldrich), and 4% (w/v) citric acid (Sigma-Aldrich) in pure, anhydrous ethanol (Koptec. King of Prussia. PA), with 5% (w/c) sodium bicarbonate (Sigma-Aldrich) suspended in the solution. PVP was chosen as the matrix material for the backing because of its solubility in ethanol during manufacturing and solubility in water during microneedle patch application to the skin. After air-drying for 1 hour, the mold was placed in a desiccator overnight at room temperature (20° C.-25° C.) for complete drying, after which a layer of adhesive paper was gently attached onto the top surface of the PDMS mold and was used to carefully peel the patch from the mold. The demolded microneedle patches were stored in a desiccator until use.

These microneedle patches, comprising Nile red dye, were inserted by pressing into the skin ex vivo from pigs with a thumb for 10 seconds, then waiting another 50 seconds without pressure for the dissolution of the effervescent backing and separation of the microneedles from the patch backing. After removal of the patch backing, the skin containing implanted microneedles was examined by optical microscopy (Olympus, Tokyo, Japan) to identify the detached microneedles embedded in the skin. In some cases, a swab was gently and repeatedly scraped across the site of the microneedle patch treatment for 10 seconds to remove any detached microneedles that were partially protruding above the skin surface.

After application to the skin for 1 minute, about 95% of the microneedles per patch separated from the patch backing and were retained in skin and over 90% of the model drug (fluorescent dye) was delivered to the skin. Penetration depth of the core-shell microneedles was approximately 250 μm into the skin after separation from the patch backing. This demonstrates effective microneedle detachment and delivery in skin using core-shell microneedle patches with an effervescent patch backing.

To assess only the penetration of the microneedle patches, patches were applied to the skin by thumb and then immediately removed. The skin was then covered with gentian violet solution (Humco, Linden, TX) for 10 minutes to stain the sites of microneedle penetration, then cleaned with alcohol swabs to remove residual dye from the skin surface.

The penetration and detaching efficiencies were calculated by dividing the number of colored spots (i.e., due to gentian violet staining or the presence of fluorescent microneedles in the skin) by the number of microneedles in the patch (i.e., 112). The delivery efficiency was calculated by dividing the amount of Nile red dye (measured by fluorescence spectrometry) in the skin (determined by subtracting the dye in the residual microneedles on the patch after use from the amount of dye in the microneedles of the patch before use) by the amount of dye in the microneedle patch before use.

### Example 5. Release of LNG From Core-Shell Microneedle Patches in Vitro

Release kinetics of LNG from core-shell microneedles in vitro was studied. The in vitro release data may reasonably predict in vivo release behavior. This is because prior work showed good in vitro to in vivo correlation of LNG release in rats from microneedles formulated with PLA and PLGA when compared to LNG release at 37° C., in release media comprised of PBS containing 20% ethanol.

To evaluate the in vitro release of LNG from microneedle patches, the release medium of PBS (137 mM NaCl, 2.68 mM KCI, 10.14 mM Na₂HPO₄, 1.76 mM KH₂PO₄) was used with 20% (w/v) ethanol added to better mimic in vivo release kinetics of other PLGA-based microneedles in rats. Specifically, one microneedle patch was placed into 1 L of release medium in a glass vessel. The glass vessel was incubated in a shaker water bath at 37°° C., that was shaken at 80 rpm. At pre-determined time points (0. 1, 3, 7, 14, 21, 28 days, and every 7 days after that until reaching the point of 182 days), 1 mL release medium was collected and replaced with the same amount of fresh medium.

Collected samples were analyzed by UPLC (Acquity, Waters Corp., Milford, MA) equipped with a UV detector to quantify the LNG concentration. LNG was separated on an Acquity UPLC Ethylene-Bridged Hybrids (BEH) C18 column (100 mm×2.1 mm i.d.; 1.7 μm particle size) at 50° C. A mixture of acetonitrile containing 0.1% formic acid, and water containing 0.1% formic acid (55:45 ratio, v/v) comprised the mobile phase. The injection volume was 10 μL, with the flow rate of 0.3 mL min⁻¹. The UV absorbance of LNG was measured at 245 nm.

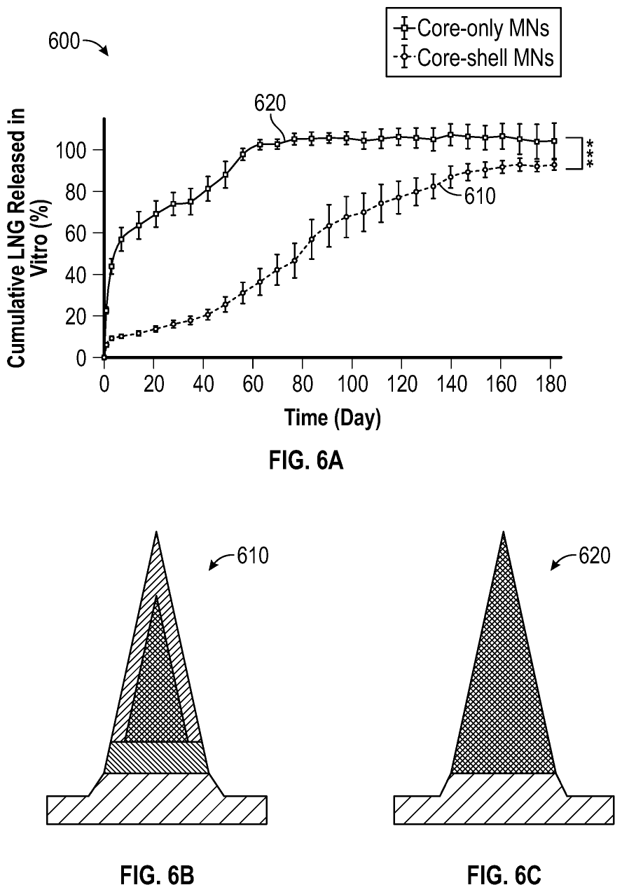

Cumulative LNG release (600) was plotted over time for the 182-day study, as shown in FIG. 6A. Because residual LNG remained in the microneedles at the end of the study, the release curve was extrapolated by continuing it at the average release rate until 100% release was achieved, and reported this value as the duration of LNG release.

Core-only microneedle patches 620, e.g., FIG. 6C, exhibited a 22.6±2.0% burst release of LNG on day one, whereas burst release from core-shell microneedle patches 610, e.g., FIG. 6B, was just 5.8±0.5% LNG on day one, indicating that LNG encapsulation in the shell-cap pocket significantly reduced burst release (two-tailed Student's t-test, p<0.001).

The monolithic core-only microneedle patches 620 achieved a typical first-order release of LNG for 2.1±0.2 months, while the core-shell microneedles 610 achieved significantly longer and closer to zero-order release profile for 6.2±0.1 months (two-tailed Student's t-test, p<0.001). The average LNG release rate for core-only microneedle patches 620 was 1.6±0.3% per day, which was significantly faster than for the core-shell microneedle patches 610 that released 0.6±0.2% of LNG per day (two-tailed Student's t-test, p<0.01). Altogether, these data demonstrate that the core-shell microneedle patch 610 effectively encapsulated LNG within a rate-controlling shell-cap pocket to significantly reduce the drug release rate and achieve 6-month release kinetics.

Some embodiments of the present disclosure can be described in view of one or more of the following:
Embodiment 1. A microneedle patch comprising: a backing layer; and an array of microneedles extending from the backing layer, the microneedles each comprising; a core portion which comprises a drug, and a shell portion and a cap portion, wherein the core portion is encapsulated within the shell portion and cap portion, wherein the microneedles are configured to be inserted into mammalian tissue, and separate from the backing layer, and wherein the shell portion and/or the cap portion is biodegradable and delays release of the drug for an extended period.
Embodiment 2. The microneedle patch of Embodiment 1, wherein the microneedles are configured to release the drug via diffusion through the shell portion only, through the cap portion only, or through both the shell portion and the cap portion.
Embodiment 3. The microneedle patch of Embodiment 1 or 2, wherein the shell portion comprises polylactide (PLA) or poly (L-lactide) (PLLA).
Embodiment 4. The microneedle patch of any one of Embodiments 1 to 3, wherein the cap portion comprises polylactide (PLA) or poly (L-lactide) (PLLA).
Embodiment 5. The microneedle patch of any one of Embodiments 1 to 4, wherein the backing layer is formed of a water-soluble polymer.
Embodiment 6. The microneedle patch of any one of Embodiments 1 to 5, wherein the backing layer comprises polyvinyl pyrrolidone (PVP), polyvinyl alcohol (PVA), sucrose, or a combination thereof.
Embodiment 7. The microneedle patch of any one of Embodiments 1 to 6, wherein the backing layer comprises sodium bicarbonate, citric acid, and/or other effervescent materials.
Embodiment 8. The microneedle patch of any one of Embodiments 1 to 7, wherein the core portion comprises a biodegradable polymer in which the drug is dispersed.
Embodiment 9. The microneedle patch of claim 8, wherein the biodegradable polymer of the core portion comprises poly (lactide-co-glycolide) (PLGA).
Embodiment 10. The microneedle patch of any one of Embodiments 1 to 9, further comprising a pedestal between the backing layer and each of the cap portions of the microneedles.
Embodiment 11. The microneedle patch of Embodiment 10, wherein the pedestal is integrally formed with the backing layer.
Embodiment 12. The microneedle patch of any one of Embodiments 1 to 11, wherein the microneedles are configured to separate from the backing layer by a process that comprises (i) aqueous dissolution of a material forming at least part of the backing layer, the material optionally including an effervescent material, and/or (ii) fracture at an interface of the cap portion and the backing layer.
Embodiment 13. The microneedle patch of Embodiment 12, wherein the fracture is caused by an air pocket at the interface of the cap portion and the backing layer.
Embodiment 14. The microneedle patch of Embodiment 12 or 13, wherein the cap portion comprises a hydrophobic material and the backing layer comprises a hydrophilic, such that the interface of the cap portion and backing layer is weak and susceptible to fracture.
Embodiment 15. The microneedle patch of any one of Embodiments 1 to 14, wherein the microneedles are configured to separate within a period of 1 minute or less after the microneedles are inserted into mammalian tissue.
Embodiment 16. The microneedle patch of any one of Embodiments 1 to 15, which is configured to release the drug from the separated microneedle continuously over a period from 1 month week to 1 year.
Embodiment 17. The microneedle patch of any one of Embodiments 1 to 16, which is configured to release the drug from the separated microneedle continuously over a period from 2 months to 6 months.
Embodiment 18. The microneedle patch of any one of Embodiments 1 to 17, where the drug comprises a steroid.
Embodiment 19. The microneedle patch of any one of Embodiments 1 to 17, where the drug comprises a hormone, such as levonorgestrel, etonogestrel, nestorone or another contraceptive hormone.
Embodiment 20. The microneedle patch of any one of Embodiments 1 to 17, wherein the drug is effective to treat a dermatological condition.
Embodiment 21. The microneedle patch of any one of Embodiments 1 and 3 to 15, where the drug comprises a vaccine.
Embodiment 22. The microneedle patch of any one of Embodiments 1 to 21, where the shell portion and/or the cap portion comprise a drug that is the same as or different from the drug in the core portion.
Embodiment 23: A microneedle patch for administering a contraceptive agent or other drug, the patch comprising: a backing layer comprising an array of pedestals; and an array of microneedles extending from each of the pedestals, the microneedles each comprising: a core portion which comprises a biodegradable polymer in which a contraceptive agent or other drug is dissolved or dispersed, and a water insoluble, and preferably non-porous, shell portion and cap portion, wherein the core portion is enclosed within the shell portion and cap portion, wherein the microneedles are configured to be inserted into mammalian tissue, separate from the pedestals, and then release an effective amount of the contraceptive agent or other drug via diffusion through the shell portion only, through the cap portion only, or through both the shell portion and the cap portion, over an extended period of at least 30 days.
Embodiment 24. The microneedle patch of Embodiment 23, wherein the shell portion and the cap portion are formed of biodegradable polymers.
Embodiment 25. The microneedle patch of Embodiment 23 or 24, wherein the shell portion comprises poly (L-lactide) (PLLA), and wherein the cap portion comprises polylactic acid (PLA).
Embodiment 26. The microneedle patch of any one of Embodiments 22 to 25, wherein the water-soluble polymer comprises polyvinyl pyrrolidone (PVP), polyvinyl alcohol (PVA), sucrose, or a combination thereof.
Embodiment 27. The microneedle patch of any one of Embodiments 22 to 26, wherein the extended period is from 2 months to 6 months.
Embodiment 28. The microneedle patch of any one of Embodiments 22 to 27, wherein contraceptive agent comprises levonorgestrel, etonogestrel, nestorone, or another contraceptive hormone.
Embodiment 29. A method of making a microneedle patch, the method comprising: (i) forming a plurality of microneedles in a mold, wherein each microneedle has a core portion comprising a drug, and a shell portion and a cap portion that fully surround the core portion; (ii) forming a backing layer connected to a base end of each of the plurality of microneedles; and then (iii) removing the backing layer and microneedles from the mold, thereby producing a microneedle patch.
Embodiment 30. The method of Embodiment 29, wherein the backing layer comprises an array of pedestals, each being connected to the base end of one or more of the plurality of microneedles.
Embodiment 31. The method of Embodiment 29 or 30, wherein the microneedles are configured to release the drug via diffusion from the core portions through the shell portions and/or through the cap portions of the microneedles.
Embodiment 32. A method comprising: (i) casting a first composition in a mold having cavities defining a plurality of microneedles to form a shell portion in the mold; (ii) casting a second composition, which comprises a drug, into an interior space defined by the shell portion in the mold to form a core portion; (iii) casting a third composition onto the core portion in the mold to form a cap portion; and (iv) casting a fourth composition onto the cap portion in the mold to form a backing layer, thereby producing a microneedle patch comprising an array of microneedles extending from the backing laver, wherein each of the microneedles comprises the core portion, which comprises the drug, enclosed within the shell portion and the cap portion.
Embodiment 33. The method of Embodiment 32, wherein the first, second, third, and fourth compositions each comprises a fluid comprising a polymeric structural material dissolved in a solvent, wherein the solvent of the first composition differs from at least the solvent of the second composition, the solvent of the second composition differs from at least the solvent of the first and third compositions, and the solvent of the third composition differs from at least the solvent of the second and fourth compositions, and the solvent of the fourth composition differs from the solvent of at least the solvent of the third composition.
Embodiment 34. The method of Embodiment 33, wherein the polymeric structural material of the first, second, and third compositions each comprises a biodegradable polymer.
Embodiment 35. The method of Embodiment 33 or 34, wherein the polymeric structural material of fourth composition comprises a water-soluble polymer.
Embodiment 36. The method of any one of Embodiment 32 to 35, wherein the first composition comprises PLLA dissolved in dioxane.
Embodiment 37. The method of any one of Embodiment 32 to 36, wherein the second composition comprises PLGA dissolved in a mixture of diglyme and water.
Embodiment 38. The method of any one of Embodiment 32 to 37, wherein the third composition comprises PLA dissolved in dimethylacetamide.
Embodiment 39. The method of any one of Embodiment 32 to 38, wherein the fourth composition comprises PVA and sucrose dissolved in water.
Embodiment 40. The method of any one of Embodiment 32 to 39, wherein the fourth composition comprises PVP, sodium bicarbonate, and citric acid dispersed or dissolved in anhydrous ethanol.
Embodiment 41. The method of any one of Embodiment 32 to 40, further comprising centrifuging the mold following the casting of the first composition to facilitate formation of a film in the mold that will become the shell.
Embodiment 42. The method of any one of Embodiment 32 to 41, further comprising drying contents of the mold following each of the castings.
Embodiment 43. The method of any one of Embodiment 32 to 42, further comprising removing the microneedle patch from the mold.
Embodiment 44. A method of administering a drug to a patient, the method comprising: (i) inserting the microneedles of the microneedle patch of any one of the preceding Embodiments into a patient's skin; (ii) separating the microneedles from the backing layer; and then (iii) releasing the drug from the core portions of the microneedles, wherein the shell portion and/or the cap portion is biodegradable and delays release of the drug for an extended period, optionally wherein the release is via diffusion through the shell portions of the microneedles.
Embodiment 45. The method of Embodiment 44, further comprising releasing the drug from the core portions via diffusion through the cap portions of the microneedles.
Embodiment 46. The method of Embodiment 44 or 45, wherein the drug is released continuously over a period of at least one month.
Embodiment 47. The method of Embodiment 46, wherein the period is from 2 months to 6 months.
Embodiment 48. The method of any one of Embodiments 44 to 47, wherein the drug comprises levonorgestrel, etonogestrel, nestorone, or another contraceptive hormone.

The term “about,” as used herein, indicates the value of a given quantity and can include quantities ranging within 10% of the stated value, or optionally, within 5% of the value, or in some embodiments, within 1% of the value.

While the disclosure has been described with reference to a number of exemplary embodiments, it would be understood by those skilled in the art that the disclosure is not limited to such disclosed embodiments. Rather, the disclosed embodiments can be modified to incorporate any number of variations, alterations, substitutions, or equivalent arrangements not described herein, but which are commensurate with the spirt and scope of the disclosure.

1. A microneedle patch comprising:
a backing layer; and
an array of microneedles extending from the backing layer, the microneedles each comprising:
a core portion which comprises a drug, and
a shell portion and a cap portion, wherein the core portion is encapsulated within the shell portion and cap portion,
wherein the microneedles are configured to be inserted into mammalian tissue, and separate from the backing layer, and
wherein the shell portion and/or the cap portion is biodegradable and delays release of the drug for an extended period.

2. The microneedle patch of claim 1, wherein the microneedles are configured to release the drug via diffusion through the shell portion only, through the cap portion only, or through both the shell portion and the cap portion.

3. The microneedle patch of claim 1, wherein the shell portion and/or the cap portion comprises polylactide (PLA) or poly (L-lactide) (PLLA).

4. (canceled)

5. The microneedle patch of claim 1, wherein the backing layer is formed of a water-soluble polymer.

6. The microneedle patch of claim 5, wherein the backing layer comprises polyvinyl pyrrolidone (PVP), polyvinyl alcohol (PVA), sucrose, or a combination thereof.

7. The microneedle patch of claim 5, wherein the backing layer comprises sodium bicarbonate, citric acid, and/or other effervescent materials.

8. The microneedle patch of claim 1, wherein the core portion comprises a biodegradable polymer in which the drug is dispersed.

9. The microneedle patch of claim 8, wherein the biodegradable polymer comprises poly (lactide-co-glycolide) (PLGA).

10. The microneedle patch of claim 1, further comprising a pedestal between the backing layer and each of the cap portions of the microneedles.

11. The microneedle patch of claim 10, wherein the pedestal is integrally formed with the backing layer.

12. The microneedle patch of claim 1, wherein the microneedles are configured to separate from the backing layer by a process that comprises (i) aqueous dissolution of a material forming at least part of the backing layer, the material optionally including an effervescent material, and/or (ii) fracture at an interface of the cap portion and the backing layer.

13. The microneedle patch of claim 12, wherein the fracture is caused by an air pocket at the interface of the cap portion and the backing layer.

14. The microneedle patch of claim 12, wherein the cap portion comprises a hydrophobic material and the backing layer comprises a hydrophilic, such that the interface of the cap portion and backing layer is weak and susceptible to fracture.

15. The microneedle patch of claim 12, wherein the microneedles are configured to separate within a period of 1 minute or less after the microneedles are inserted into mammalian tissue.

16. The microneedle patch of claim 1, which is configured to release the drug from the separated microneedle continuously over a period from 1-month week to 1 year.

17. (canceled)

18. The microneedle patch of claim 1, where the drug comprises a steroid.

19. (canceled)

20. The microneedle patch of claim 1, wherein the drug is effective to treat a dermatological condition.

21. The microneedle patch of claim 1, where the drug comprises a vaccine.

22. The microneedle patch of claim 1, where the shell portion and/or the cap portion comprise a drug that is the same as or different from the drug in the core portion.

23. A microneedle patch for administering a contraceptive agent or other drug comprising:
a backing layer comprising an array of pedestals; and
an array of microneedles extending from each of the pedestals, the microneedles each comprising:
a core portion which comprises a biodegradable polymer in which a contraceptive agent or other drug is disperse, and
a water insoluble shell portion and cap portion, wherein the core portion is enclosed within the shell portion and cap portion,
wherein the microneedles are configured to be inserted into mammalian tissue, separate from the pedestals, and then release an effective amount of the contraceptive agent or other drug via diffusion through the shell portion only, through the cap portion only, or through both the shell portion and the cap portion, over an extended period of at least 30 days.

24. The microneedle patch of claim 23, wherein the shell portion and the cap portion are formed of biodegradable polymers.

25. The microneedle patch of claim 24, wherein the shell portion comprises poly (L-lactide) (PLLA), and wherein the cap portion comprises polylactic acid (PLA).

26. The microneedle patch of claim 23, wherein the water-soluble polymer comprises polyvinyl pyrrolidone (PVP), polyvinyl alcohol (PVA), sucrose, or a combination thereof.

27. The microneedle patch of claim 23, wherein the extended period is from 2 months to 6 months.

28. The microneedle patch of claim 23, wherein contraceptive agent comprises levonorgestrel, etonogestrel, nestorone, or another contraceptive hormone.

29. A method of making a microneedle patch, the method comprising:
forming a plurality of microneedles in a mold, wherein each microneedle has a core portion comprising a drug, and a shell portion and a cap portion that fully surround the core portion;
forming a backing layer connected to a base end of each of the plurality of microneedles; and then
removing the backing layer and microneedles from the mold, thereby producing a microneedle patch.

30-31. (canceled)

32. A method comprising:
casting a first composition in a mold having cavities defining a plurality of microneedles to form a shell portion in the mold;
casting a second composition, which comprises a drug, into an interior space defined by the shell portion in the mold to form a core portion;
casting a third composition onto the core portion in the mold to form a cap portion; and
casting a fourth composition onto the cap portion in the mold to form a backing layer,
thereby producing a microneedle patch comprising an array of microneedles extending from the backing layer, wherein each of the microneedles comprises the core portion, which comprises the drug, enclosed within the shell portion and the cap portion.

33-43. (canceled)

44. A method of administering a drug to a patient, the method comprising:
inserting the microneedles of the microneedle patch of claim 1 into a patient's skin;
separating the microneedles from the backing layer;
permitting the shell portion and/or the cap portion to biodegrade in vivo; and
releasing the drug over an extended period.

45-49. (canceled)
